(19) United States
(12) Patent Application Publication
(10) Pub. No.: US 2002/0197639 A1
(43) Pub. Date: Dec. 26, 2002

(54) Methods and products for analyzing nucleic acids based on methylation status

(76) Inventors: Michael A. Shia, Cambridge, MA (US); Gordon G. Wong, Brookline, MA (US)

Correspondence Address: Maria A. Trevisan, Wolf, Greenfield & Sacks, P.C., Federal Reserve Plaza, 600 Atlantic Avenue, Boston, MA, 02210, US

(21) Appl. No.: 10/165,914

(22) Filed: Jun. 10, 2002

Related U.S. Application Data
(60) Provisional application No. 60/297,147, filed on Jun. 8, 2001.

(51) Int. Cl. C12Q 1/68; C12P 19/34

(52) U.S. Cl. 435/6; 435/91.2

(57) ABSTRACT

The invention relates to methods, products and systems for analyzing nucleic acid molecules based on their in vivo methylation status. The methods can be used to obtain sequence information about the nucleic acid molecules, to analyze differential gene expression associated with disorders, and to assess the efficacy of therapeutic treatments that affect methylation status.

### RELATED APPLICATIONS

This application claims priority to U.S. Provisional Patent Application filed Jun. 8, 2002, entitled “METHODS AND PRODUCTS FOR ANALYZING NUCLEIC ACIDS BASED ON METHYLATION STATUS”, Serial No. 60/297,147, the contents of which are incorporated by reference herein in their entirety.

### FIELD OF THE INVENTION

The invention relates to analysis of the methylation status of nucleic acids, and to the exploitation of methylation mechanisms to sequence nucleic acids.

### BACKGROUND OF THE INVENTION

It is known that nucleic acids are methylated. Methylation of DNA is involved in both normal and abnormal cellular processes. For example, DNA methylation has been implicated in X-inactivation, imprinting of parental alleles, and differential gene expression (either by upregulation or silencing of genetic loci). In bacteria, methylation of cytosine and adenine residues plays a role in the regulation of DNA replication and DNA repair. DNA methylation also constitutes part of a immune mechanism that allows these organisms to distinguish between self and non-self DNA. DNA methylation has also been associated with increased risk of cancer, as well as cancer development itself.

Methylation of DNA is carried out by methylases (also known as methyltransferases). These enzymes are generally sequence-specific, and they can methylate both nucleic acid strands (in the case of DNA). Replication of these strands yields a hemi-methylated state which is recognized by a class of maintenance methylases capable of restoring full methylation to both strands.

Methylation can occur at all nucleotide residues, although in mammalian species, DNA methylation commonly occurs at cytosine residues, and more commonly at cytosine residues that lie next to a guanosine residue, i.e., at cytosine residues of a CG dinucleotide. CG dinucleotides in “CpG islands” remain methylation-free. CpG islands are rich in CG sites and are often found near coding regions within the genome (i.e., genes). About half of the genes in the human genome are associated with CpG islands. Importantly, the vast majority of CpG islands in the genome remain unmethylated in normal adult cells and tissues. Methylation of CpG islands is normally seen only on the inactive X-chromosome in females and at imprinted genes where it functions in the stable silencing of such genes. Strict control over the levels and distribution of DNA methylation are essential to normal animal development.

Alteration in DNA methylation is one manifestation of the genome instability characteristic of human tumors. A hallmark of human carcinogenesis is the loss of normal constraints on cell growth resulting from genetic alterations in the genes that control cell growth. The consequences of such mutations include the activation of positive growth signals and the inactivation of growth inhibitory signals. Identification of gene targets which when methylated lead to the loss of normal cell responses would be valuable. This would facilitate the diagnosis and treatment of disorders associated with abnormal methylation and any downstream events resulting therefrom.

The level of methylation of a nucleic acid can be determined using a number of techniques available in the art. Some indirect methods of analysis involve the use of bisulfite to deaminate and convert methylated cytosines to uracils. Upon amplification, the uracils are then effectively synthesized with the complementary adenosine. This synthesis thus allows for analysis of the methylated sites via sequencing or hybridization-based approaches to determine the locations of the methylated sites on the strand of DNA. Prior art methods for methylation analysis include methylation-sensitive restriction analysis, methylation-specific polymerase chain reaction (MSP), sequencing of bisulfite-modified DNA, Ms-SnuPE, and COBRA.

Typically, direct analysis of a methylation pattern on a single nucleic acid molecule is not possible. Thus, methods for direct detection of methylation of a nucleic acid molecule would be useful, as would methods for determining number, type and location of methylation within a single nucleic acid molecule.

### SUMMARY OF THE INVENTION

The invention is premised on the observation that the methylation status of nucleic acids can be analyzed. Methylation status of a nucleic acid molecule imparts information relating to imprinting of alleles, gene expression and silencing, and identification of genomic mutation, for example. The invention also exploits methylation machinery and processes in order to derive sequence information about single nucleic acid molecules.

In one aspect, the invention provides a method for analyzing a nucleic acid molecule, comprising exposing a nucleic acid molecule to a sequence-specific methylase and an S-adenosyl methionine (SAM) labeled derivative, allowing the sequence-specific methylase to label nucleotides in the nucleic acid molecule with the SAM labeled derivative, and determining the labeling pattern in the nucleic acid molecule using a linear polymer analysis system.

In all aspects disclosed herein, the nucleic acid molecule may be DNA or RNA, or some combination thereof. It may be naturally occurring, and possibly harvested from in vivo sources, or synthesized in vitro. It may further have a phosphodiester backbone, or may contain backbone modifications such as those discussed herein. In some important embodiments, the nucleic acid molecule is a non in vitro amplified nucleic acid molecule.

In this and other aspects of the invention, the nucleic acid molecule may be exposed to a demethylating agent such as a demethylating enzyme in an amount effective to demethylate the nucleic acid molecule. This exposure may occur prior to or following exposure to an agent such as the sequence-specific methylase and the SAM labeled derivative mentioned above, or a methylation-specific antibody or antibody fragment, or a methylated nucleic acid binding protein (MBP), as described below. In some embodiments, a methylation pattern prior to exposure to a demethylating agent, and a methylation pattern after exposure to a demethylating agent and a re-labeling process, are compared and differences in the methylation patterns are identified. These differences can be quantitative in terms of the level or frequency of methylation, and/or qualitative in terms of the location or type of methylation.

In another aspect, the invention provides a related method for analyzing a nucleic acid molecule, wherein the sequence specific methylase is labeled and the SAM derivative is not. In important embodiments, the SAM derivative is an aziridine derivative. The methylase may be labeled using any of the labels described herein for labeling the SAM derivative. In important embodiments, the methylase comprises a plurality of labels, which may be identical or different, and which may be of identical or different type, as described herein.

In yet another related aspect, another method is provided for analyzing a nucleic acid molecule, wherein SAM is used rather than a SAM derivative, and the SAM is itself labeled, with for example a radioactive label.

In another aspect of the invention, a method is provided for analyzing a nucleic acid molecule, comprising exposing the nucleic acid molecule to a methylation-specific antibody or antibody fragment, allowing the antibody or antibody fragment to bind to the nucleic acid molecule, and determining a binding pattern of the methylation-specific antibody or antibody fragment to the nucleic acid molecule using a linear polymer analysis system, such as but not limited to those described herein. The antibody or fragment thereof is preferably labeled with a detectable label. Alternatively, the antibody or fragment thereof is detected by use of a secondary label that recognizes and binds to the antibody and which itself may be a detectable label or may have bound to it a detectable label.

In one embodiment, the methylation-specific antibody or antibody fragment recognizes and binds to methylated nucleotides selected from the group consisting of methylated adenosine, methylated cytosine, methylated guanosine, methylated thymine, and methylated uridine. In other embodiments, the methylation-specific antibody or antibody fragment recognizes and binds to 6-methyladenosine, 4-methylcytosine and 5-methylcytosine. Other methylated nucleotides can also be detected using the methods of the invention and these include both “normal” methylated nucleotides (i.e., nucleotide methylations that occur normally in cells for example, in order to silence expression from a gene locus) and mutagenic methylated nucleotides (i.e., nucleotide methylations that occur following exposure to a DNA damaging agent and which are associated with a particular disorder such as cancer). Other methylated nucleotides that can be detected include 7-methylguanine, O⁴-methylthymine, O⁶-methylguanine, 2,2,7-trimethylguanine, and the like.

In a related aspect, the methylation pattern of a nucleic acid molecule is determined using a MBP. The method comprises exposing a nucleic acid molecule to a MBP, allowing the MBP to bind to the nucleic acid molecule, and determining the binding pattern of the MBP to the nucleic acid molecule. In some embodiments, the MBP is labeled with a detectable label. In other embodiments, the nucleic acid molecule is treated with a demethylating agent following binding to the MBP and then labeled with a sequence-specific methylase and a SAM derivative or SAM as described above.

Depending upon the embodiment, exposure to the demethylating agent, with methylation or SAM derivative labeling thereafter, can be is used to determine the total methylation sites in a nucleic acid molecule. Thus, in one embodiment, after determining the methylation pattern in the nucleic acid molecule (e.g., using any of the foregoing methods), the nucleic acid molecule is exposed to a demethylating agent (e.g., a demethylating enzyme) in an amount effective to demethylate the nucleic acid molecule. The nucleic acid molecule is then exposed to and labeled with a sequence-specific methylase and a SAM labeled derivative or SAM.

Thus, in one embodiment, the method further comprises, after determining the binding pattern of the methylation-specific antibody or antibody fragment, exposing the nucleic acid molecule to a demethylating agent (e.g., a demethylating enzyme) in an amount effective to demethylate the nucleic acid molecule, and re-exposing the nucleic acid molecule to a sequence-specific methylase and a SAM labeled derivative, allowing the sequence-specific methylase to modify target nucleotides in the nucleic acid molecule with the SAM labeled derivative, and determining the methylation pattern.

In the foregoing aspects, the methylation patterns before and after demethylation can be compared to normal methylation patterns or to genomic maps.

In certain embodiments, the SAM labeled derivative comprises a label selected from the group consisting of a fluorescent molecule, a chemiluminescent molecule, a radioisotope, an enzyme substrate, a biotin molecule, an avidin molecule, an electrically charged transducing molecule, a nuclear magnetic resonance molecule, a semiconductor nanocrystal, an electromagnetic molecule, an electrically conducting particle, a ligand, a microbead, a magnetic bead, a Qdot, a chromogenic substrate, an affinity molecule, a protein, a peptide, a nucleic acid, a carbohydrate, an antigen, a hapten, an antibody, an antibody fragment, and a lipid. As discussed herein, methylation-specific antibody and antibody fragments, and MBPs may also be labeled with the above labels. The methods described herein may further comprise labeling the nucleic acid molecule with a backbone label.

In one embodiment, the pattern of nucleotide modification in the nucleic acid molecule is determined using a linear polymer analysis system such as the Gene Engine™ system, optical mapping, DNA combing, and the like.

In one embodiment, the nucleic acid molecule is exposed to a station to produce a signal arising from the nucleotide modification, and detecting the signal using a detection system. In some embodiments, the nucleic acid molecule is attached to a solid-support, while in others, it is free in solution.

In related embodiments, the detection system is selected from the group consisting of a fluorescent detection system, an electrical detection system, a photographic film detection system, a chemiluminescent detection system, an enzyme detection system, an atom force microscopy (AFM) detection system, a scanning tunneling microscopy (STM) detection system, an optical detection system, a nuclear magnetic resonance (NMR) detection system, a near field detection system, a total internal reflection (TIR) system, and a electromagnetic detection system.

In yet another aspect, the invention provides a method for identifying a subject having or at risk of developing a disorder characterized by abnormal methylation of a nucleic acid molecule, comprising determining a methylation pattern of a nucleic acid molecule in a biological sample from a subject, and comparing the methylation pattern of the nucleic acid molecule to a control, wherein a difference in the methylation pattern of the nucleic acid molecule as compared to the control identifies a subject having or at risk of developing a disorder. In this and other aspects of the invention, the subject may be human.

In one embodiment, the methylation pattern is determined by exposing the nucleic acid molecule to a methylation-specific antibody or antibody fragment, or a MBP, and determining the binding pattern to the nucleic acid molecule using a linear polymer analysis system. In another embodiment, the methylation pattern is determined by exposing the nucleic acid molecule to a sequence-specific methylase and a SAM labeled derivative, allowing the sequence-specific methylase to label nucleotides in the nucleic acid molecule with the SAM labeled derivative, and determining the labeling pattern in the nucleic acid molecule.

In one embodiment, the control is a normal cell, or a normal tissue sample. In another embodiment, the control is a set of data from normal cells.

In another aspect, the invention provides a method for assessing the efficacy of a therapeutic treatment, comprising determining a methylation pattern of a nucleic acid molecule in a biological sample from a subject prior to and after the therapeutic treatment, and comparing the methylation pattern prior to the therapeutic treatment with the pattern of methylation after the therapeutic treatment, wherein a difference in the methylation pattern as a result of the therapeutic treatment is an indicator of the efficacy of the therapeutic treatment.

In one embodiment, the difference in the methylation pattern is an increase in a total level of methylation. In another embodiment, the difference in the methylation pattern is a decrease in a total level of methylation. In yet other embodiments, the difference in the methylation pattern is a difference in the location of methylation, or a difference in the frequency of methylation in a defined location, or a difference in the type of methylation.

In one embodiment, the therapeutic treatment is an anti-cancer agent. In another embodiment, the therapeutic treatment includes administration of an inhibitor of methyltransferase. The inhibitor of methyltransferase may be selected from the group consisting of 5-azacytidine, 5-aza-2′deoxycytidine, 5, 6-dihydro-5-azacytidine, 5-fluorocytidine and 5fluoro-2′deoxycytidine, but is not so limited.

In a further aspect, the invention provides a system for optically analyzing a nucleic acid molecule comprising an optical source for emitting optical radiation of a known wavelength; an interaction station for receiving the optical radiation in an optical path and for receiving the nucleic acid molecule that is exposed to the optical radiation to produce detectable signals; dichroic reflectors in the optical path for creating at least two separate wavelength bands of the detectable signals; optical detectors constructed to detect radiation including the signals resulting from interaction of the nucleic acid molecule with the optical radiation; and a processor constructed and arranged to analyze the nucleic acid molecule based on the detected radiation including the signals, wherein the nucleic acid molecule is labeled according to its methylation status.

In one embodiment, the interaction station includes a localized radiation spot. In a further embodiment, the system further comprises a microchannel that is constructed to receive and advance the polymer units through the localized radiation spot. In another embodiment, the system further comprises a polarizer, wherein the optical source includes a laser constructed to emit a beam of radiation and the polarizer is arranged to polarize the beam. In some embodiments, the localized radiation spot is produced using a slit. The slit may have a slit width in the range of 1 nm to 500 nm, or in the range of 10 nm to 100 nm. In some embodiments, the polarizer is arranged to polarize the beam prior to reaching the slit. In other embodiments, the polarizer is arranged to polarize the beam in parallel to the width of the slit.

The nucleic acid molecules analyzed using the linear polymer analysis systems, such as the foregoing system, are labeled using any of the methods described herein, as well as combinations thereof.

In one aspect of the invention, a method is provided for analyzing a nucleic acid molecule comprising generating optical radiation of a known wavelength to produce a localized radiation spot; passing a labeled nucleic acid molecule through a microchannel; irradiating the labeled nucleic acid molecule at the localized radiation spot; sequentially detecting radiation resulting from interaction of the labeled nucleic acid with the optical radiation at the localized radiation spot; and analyzing the labeled nucleic acid molecule based on the detected radiation, wherein the nucleic acid molecule is labeled according to its methylation status.

In one embodiment, the method further comprises employing an electric field to pass the nucleic acid molecule through the microchannel. In another embodiment, the detecting includes collecting the signals over time while the nucleic acid molecule is passing through the microchannel.

The labeling methods of the invention can employ a) labeled sequence specific methylases and labeled SAM derivatives, b) labeled sequence specific methylase and SAM derivatives that are not labeled, c) sequence specific methylases and labeled SAM derivatives or labeled SAM, and d) sequence specific methylases and SAM, neither of which is labeled. In these latter embodiments, the methylation must be detected through the use of a methylation specific antibody or fragment, or a MBP. In important embodiments, the SAM derivative is an aziridine derivative. The sequence-specific methylase, SAM derivatives, and SAM can be labeled with any of the detectable labels described herein.

These and other embodiments of the invention will be discussed in greater detail herein.

### DETAILED DESCRIPTION OF THE INVENTION

Single nucleic acid molecule detection allows the direct interrogation of structural motifs on a single nucleic acid molecule. Direct analysis allows the methylation status of a single nucleic acid molecule to be determined. According to the invention, the identification of methylated sites on a nucleic acid molecule can be accomplished through a number of different methods. These methods include direct labeling of methylated sites on a nucleic acid molecule (using agents that recognize and bind to methylated nucleotides), as well as enzymatic modification of nucleic acid molecules resulting in labeled nucleotides. Direct labeling methods may use methylated nucleic acid binding proteins (MBPs), or methylation-specific antibodies or antibody fragments. Enzymatic modification methods may use labeled methylation cofactors (or cofactor derivatives that may be covalently and irreversibly attached to a nucleic acid molecule at methylation sites). Enzymatic methods can be used to methylate a nucleic acid molecule, or to label the nucleic acid molecule. Demethylation can also be included as part of the analysis, as can subtraction of methylation patterns determined prior to and after demethylation.

Methylation status imparts information relating to a cell or tissue from which the nucleic acid molecule derives. For example, the presence of mutagenic methylated nucleotides can indicate that the cell has been exposed to a carcinogen such as a DNA damaging agent. Certain methylation patterns may be associated with a pre-malignant or malignant state, and thus, identification of such methylation patterns prior to, for example, tumor development, can identify subjects in need of monitoring or treatment.

The ability to detect methylation within single nucleic acid molecules can also be exploited to derive sequence information from nucleic acid molecules. The use of “sequence-specific” methylases to methylate or label nucleic acid molecules in a sequence dependent manner, and the ability to detect the location and number of resulting methylated or labeled nucleotides provides a way to sequence nucleic acid molecules.

In one aspect, the invention relies on the use of sequence specific methylases and methylation substrates or substrate analogs. One methylation cofactor is S-adenosyl methionine (SAM). SAM donates a methyl group, via the action of the methylase, to another compound, such as a nucleic acid molecule. Once SAM donates the methyl group, it dissociates from the nucleic acid molecule, as does the methylase. This reaction can be used in the methods of the invention to methylate a nucleotide at a particular sequence (e.g., the recognition sequence of the sequence specific methylase). The methyl group may itself be labeled, for example, by using a tritium moiety rather than hydrogen. Other labels can also be attached to the methyl group and the invention is not intended to be limited in this manner. In the foregoing embodiments, the methylation is a reversible reaction.

If instead the methylase cofactor is a SAM derivative, such as an aziridine derivative, then the labeling of the nucleic acid can come from either or both the methylase and the SAM derivative. The SAM derivative induces an irreversible reaction by which it and optionally the methylase may become irreversibly bound to the nucleic acid molecule at or near the recognition sequence of the methylase. (In contrast, when SAM or tritiated SAM is used, the methylase is able to dissociate, and thus the reaction is referred to as a reversible one.) Since both the methylase and the SAM derivative may be irreversibly bound to the nucleic acid molecule, it is possible to label either or both in order to detect their position, and accordingly to detect the location of the methylase recognition sequence. Labeling of the methylases in some cases is preferred because more labels can be attached to the methylase than the SAM derivative. Thus, in some embodiments both the methylase and the SAM derivative are irreversibly bound.

As used herein, the methylase reaction is said to “methylate” a nucleic acid molecule if SAM or labeled SAM is used as the cofactor, and to “label” a nucleic acid molecule if a SAM derivative, whether labeled or not, is used as the cofactor.

The invention intends to embrace other SAM derivatives in addition to aziridine derivatives, particularly if such derivatives function similarly to aziridine.

It is to be understood that the methylase, antibody, and MBP methods described herein have utility in all aspects of the invention, including the mapping of sequences in a nucleic acid molecule (for the purpose of deriving sequence information), and the determination of the methylation status of a nucleic acid molecule.

In still other aspects of the invention, the methods provided herein can be used to screen methylating and demethylating activity of agents. These and other applications of the present invention are discussed herein.

Generally, the invention provides methods, compositions and systems for analyzing single nucleic acids based on methylation status. As used herein, “methylation status” refers to the level (i.e., number), location and/or type of methylated nucleotides within a nucleic acid molecule. As used herein, the terms “methylation status” and “methylation pattern” are used interchangeably. A methylation site is a sequence of contiguous linked nucleotides that is recognized and methylated by a sequence-specific methylase. A methylase is an enzyme that methylates (i.e., covalently attaches a methyl group) one or more nucleotides at a methylation site.

The methods of the invention also are not limited to the detection of particular methylated nucleotides but rather intend to capture information from all methylated nucleotides. Accordingly, the methods of the invention can be used to detect methylated adenine, methylated cytosine, methylated guanine, methylated thymine, and methylated uridine. As stated above, these methylated nucleotides can be those normally observed in normal cells, which for example are used to normally silence loci or imprint alleles. Alternatively, the methylated nucleotides can also be those that are mutagenic, meaning that they result from exposure of the nucleic acid molecule to a carcinogen such as a DNA damaging agent.

The methods intend to analyze methylation status in a variety of nucleic acid molecules.

These nucleic acid molecules include DNA and RNA, from both in vivo and in vitro sources. Methylation of mRNA, rRNA and tRNA has been reported. (Tantravahi et al. 1981, 56(3):315-320; Pope et al., 1978, 5(3):1041-1057; Liu et al. 2002, 44(11):195-204.) DNA includes genomic DNA (such as nuclear DNA and mitochondrial DNA), as well as in some instances cDNA. In important embodiments, the nucleic acid molecule is a genomic nucleic acid molecule. The nucleic acid molecules may be single stranded, double stranded, partially single stranded, and partially double stranded. As described below, the nucleic acid molecules may be naturally occurring or non naturally occurring, and additionally, their methylation status may be the result of in vivo processes, or of experimental manipulations (e.g., deliberate exposure to a putative DNA damaging agent, or a putative demethylating agent).

The term “nucleic acid” is used herein to mean multiple nucleotides (i.e. molecules comprising a sugar (e.g. ribose or deoxyribose) linked to an exchangeable organic base, which is either a substituted pyrimidine (e.g. cytosine (C), thymidine (T) or uracil (U)) or a substituted purine (e.g. adenine (A) or guanine (G)). “Nucleic acid” and “nucleic acid molecule” are used interchangeably. As used herein, the term refers to oligoribonucleotides as well as oligodeoxyribonucleotides. The term also includes polynucleosides (i.e. a polynucleotide minus a phosphate) and any other organic base containing polymer. Nucleic acid molecules can be obtained from existing nucleic acid sources (e.g., genomic or cDNA), or by synthetic means (e.g. produced by nucleic acid synthesis). The size of the nucleic acid molecule is not limiting. It can be several nucleotides in length, several hundred, several thousand, or several million nucleotides in length. In some embodiments, the nucleic acid molecule may be the length of a chromosome.

The methods of the invention may be performed in the absence of prior nucleic acid amplification in vitro. In some preferred embodiments, the nucleic acid molecule is directly harvested and isolated from a biological sample (such as a tissue or a cell culture) without the need to amplify the nucleic acid molecule. Accordingly, some embodiments of the invention involve analysis of non in vitro amplified nucleic acid molecules. As used herein, a “non in vitro amplified nucleic acid molecule” refers to a nucleic acid molecule that has not been amplified in vitro using techniques such as polymerase chain reaction or recombinant DNA methods.

A non in vitro amplified nucleic acid molecule may, however, be a nucleic acid molecule that is amplified in vivo (in the biological sample from which it was harvested) as a natural consequence of the development of the cells in the biological sample. This means that the non in vitro nucleic acid molecule may be one which is amplified in vivo as part of a gene amplification, a phenomenon that is commonly observed in some cell types and which can be associated with cancer development. As a result, the methods allow the native methylation status of nucleic acid molecules to be determined. As used herein, a “native methylation status” is the methylation status (or pattern) of a nucleic acid molecule as it exists in vivo.

Harvest and isolation of nucleic acids are routinely performed in the art and suitable methods can be found in standard molecular biology textbooks. The nucleic acid molecule may be harvested from a biological sample such as a tissue or a biological fluid. The term “tissue” as used herein refers to both localized and disseminated cell populations including brain, heart, breast, colon, bladder, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include saliva, serum, plasma, sperm, blood and urine, but are not so limited. Both invasive and non-invasive techniques can be used to obtain such samples and are well documented in the art.

In some embodiments, the invention can be used to analyze nucleic acid derivatives. As used herein, a nucleic acid derivative is a non naturally occurring nucleic acid molecule. Nucleic acid derivatives may contain non naturally occurring elements such as non naturally occurring nucleotides and backbone linkages.

Nucleic acid derivatives may include substituted purines and pyrimidines such as C-5 propyne modified bases (Wagner et al., Nature Biotechnology 14:840-844, 1996). Purines and pyrimidines include but are not limited to adenine, cytosine, guanine, thymidine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are known to those of skill in the art.

The nucleic acid derivatives may also encompass substitutions or modifications, such as in the bases and/or sugars. For example, they include nucleic acid molecules having backbone sugars which are covalently attached to low molecular weight organic groups other than a hydroxyl group at the 3′ position and other than a phosphate group at the 5′ position. Thus, nucleic acid derivatives may include a 2′-O-alkylated ribose group. In addition, nucleic acid derivatives may include sugars such as arabinose instead of ribose. The nucleic acids may be heterogeneous or homogeneous in backbone composition.

Non naturally occurring backbone linkages include but are not limited to phosphorothioate linkages, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof. The invention also embraces analysis of nucleic acid derivatives that are composed of peptide or locked nucleic acid residues.

The invention provide several ways for determining in which methylation status of nucleic acid molecules, as well as several ways of deriving sequence information from nucleic acid molecules. As used herein, the derivation of sequence information from a nucleic acid molecule is also referred to as “mapping” the nucleic acid molecule. Accordingly, the recognition sequences of the methylases are mapped onto a nucleic acid molecule, using the methods provided herein. The information obtained can be the native methylation status of the nucleic acid molecule, for example, as it existed in its natural source. This information can indicate the gene expression pattern of a cell, a potential pre-malignant or malignant phenotype, or the efficacy of a particular therapy.

The methods described herein generally involve methylating or labeling nucleic acid molecules using enzymatic means, or direct labeling of nucleic acid molecules with agents that recognize and specifically bind to methylated nucleotides. These methods can be used singly or in combination to determine methylation status or sequence information.

Some methods exploit the action of sequence-specific methylases. Methylases (or methyltransferases, as they are also called) are enzymes that covalently attach methyl groups to one or more nucleotides. Preferably, the methylases are sequence-specific. “Sequence-specific” as used herein means that the methylase recognizes a particular linear arrangement of nucleotides or derivatives thereof, and methylates either a nucleotide within that arrangement or a nucleotide in the vicinity of the arrangement. Commonly, the sequence specific methylase methylates one or more nucleotides in the same sequence it recognizes.

Sequence-specific methylases include but are not limited to SssI methylase (CpG methylase; CmG), human DNA (cytosine-5) methyltransferase (DNMT1), human DNA (cytosine-5) methyltransferase (Dnmt1) aminoterminal, DNMT3a, DNMT3b, AluI methylase (AGCmT), BamHI methylase (GCATCmC), ClaI methylase (ATCGAmT), dam methylase (GAmTC), EcoRI methylase, HaeIII methylase, HhaI methylase, HpaII methylase, MspI methylase (CmCGG), TaqI methylase, mRNA N⁶-adenosine methyltransferase (Pu(G/A)AC(U/A) with A being methylated), and rRNA methyltransferase RrmA (i.e., rRNA large subunit methyltransferase). The sequence specificity of some of the above methylases is indicated (with the methylated nucleotide indicated by an “m” following it). The sequence specificity of the other methylases as well as the nucleotides they methylate can be determined by reference to the catalogue of any commercial supplier of these enzymes including but not limited to New England Biolabs. Other methylases that can be used in the methods of the invention include the DNA repair protein, O⁶-methylguanine RNA methyltransferase, and S-adenosyl-L-methionine methyltransferase. It should also be understood that the invention embraces the use of RNA methylases as well as DNA methylases. Sequence-specific methylation of RNA has been reported in U.S. Pat. No. 5,972,705, issued Oct. 26, 1999.

Methylases are typically obtained from bacteria. Different bacterial strains have unique restriction enzyme-methyl transferase enzyme pairs. Methylation in bacterial cells is involved in defense mechanisms which allow bacterial cells to distinguish between host and foreign nucleic acids. In this latter aspect, restriction endonucleases and methylases work in conjunction to target foreign DNA for degradation. Accordingly, each known restriction endonuclease has a cognate methylase that recognizes the identical nucleic acid sequence. Each enzyme pair will recognize a unique DNA sequence. When the sequence is methylated, the restriction enzyme will not cut the nucleic acid molecule at this specific site. Bacteria methylate their own DNA at these specific recognition sites, thereby protecting their own DNA from the cognate restriction enzyme. Unmethylated DNA, such as foreign DNA that may contain this specific DNA sequence is subject the restriction endonuclease cleavage.

Methylases suitable in the methods of the invention may also derive from any number of sources, including mammalian species, nematodes such as, C. elegans, viral species, and the like.

Sequence-specific methylases commonly use SAM as a methyl donor in a standard methylation reaction. Normally, SAM will donate a methyl group to the nucleotide (resulting in a methylated nucleotide) and release from the nucleic acid molecule together with the methylase. DNA methylases can catalyze the methylation of, for example, adenine at the N⁶ position (found for example in internal positions of mRNA in higher eukaryotes), and cytosine at the C⁵ or N⁴ position. Other methylases methylate nucleic acids at different positions on these and other nucleotides.

Sequence-specific methylases can also function together with labeled derivatives of SAM of label nucleic acid molecules. PCT patent application WO 00/06587, published on Feb. 10, 2000 describes cofactors of methylases that are derivatives of SAM. SAM derivatives, when used with a methylase, will be added directly to the nucleotide that is being methylated. Unlike SAM, however, neither the substrate nor the methylase can then dissociate from the nucleic acid molecule, in the instances when the methylase becomes irreversibly bound.

As mentioned earlier, the SAM and SAM derivatives can both be labeled in order to detect their position (as in the case of SAM derivatives) or the position of the donated methyl group (as in the case of SAM). In still other embodiments, the methylase itself may be labeled, in which the SAM derivative need not be labeled (although it may, if so desired).

SAM and SAM derivatives, (e.g., aziridine derivatives) can contain reporter or other reactive chemical groups. As an example, the reporter or reactive chemical groups that can be attached to an aziridine derivative include fluorophores, reactive groups (e.g. amines, carboxyl groups, etc), affinity tags, crosslinking agents (e.g., maleimide, iodacetamide, aldehyde derivatives, photocrosslinking agents e.g., arylazide, diazo-compounds and benzophenone), chromophores, proteins (e.g., antibodies and enzymes), peptides, amino acids (modified or not), nucleotides, nucleosides, nucleic acids, carbohydrates, lipids, PEG, transfection reagents, beads (such as microbeads), and intercalating agents (e.g., ethidium bromide, psoralen, and derivatives thereof). In some embodiments, preferred fluorophores include BODIPY, coumarin, dansyl, fluorescein, mansyl, pyrene, rhodamine, Texas Red, TNS, cyanine fluorophores such as Cy2, Cy3, Cy3.5, Cy5, Cy5.5, and Cy7. Affinity tags can be selected from the group consisting of a peptide tag (e.g., his-tag, strep-tag, flag-tag, c-myc-tag, epitopes, and glutathione), biotin, digoxygenin, dinitrophenol, and the like.

One advantage of enzymatic labeling of nucleic acid molecules using methylases and a SAM derivative such as an aziridine derivative is the ability to increase the number of labels at a particular location, thereby increasing the signal generated from the modified nucleotide. This is because an the aziridine derivative induces the irreversible binding of itself and in some cases the methylase to the nucleic acid molecule. Given the number and variety of reactive groups on the surface of a protein, such as a methylase, it should be possible to introduce multiple labels into a methylase. It is not necessary to label the aziridine derivative if the methylase is labeled. Alternatively, the aziridine derivative may be labeled and the enzyme not labeled. However, in this latter embodiment, the amount of label and correspondingly the signal detected will be lower than if the methylase is labeled. In preferred embodiments, the labels on a methylase are the same, but there are instances in which they may also be different. For example, in order to increase the number of unique detectable labels available, it may be possible to combine, for example, different fluorophores in order to generate a particular unique sequence. In some embodiments, the labels on a methylase are FRET labels.

As used herein, the sequence-specific methylases together with SAM labeled derivatives “label” rather than “methylate” nucleic acid molecules. However, the labeling occurs at the same location as would methylation. Therefore, the “labeling pattern” is a surrogate for the “methylation pattern.”

The methylase reaction is carried out by using a methylase of known sequence specificity and labeled with a known label (or combination of known labels). Alternatively, the SAM or SAM derivative is labeled. Known combinations of sequence-specific methylases and labels are used so that a later incorporated label can be used as a marker of the recognition sequence of the methylase.

A series of methylase reactions may be performed consecutively. The methylation pattern may be determined between methylase reactions. Alternatively, it may be determined following the completion of all methylase reactions.

As mentioned earlier, these methods are not dependent on prior amplification of the nucleic acid molecule. Accordingly, the methylase reaction can be performed directly on freshly harvested and isolated nucleic acid molecules. Methylase reaction of the nucleic acid molecule methylates or labels those nucleotides which were unmethylated at the time of harvest. Nucleotides that were methylated at the time of harvest of the nucleic acid molecule in some instances cannot be further methylated by this procedure. However, these latter methylated sites can be determined in a number of ways described herein. These methods will be described in greater detail, however, briefly they include demethylation of all methylated sites, followed by re-methylation in order to label all sites, including those sites methylated in vivo and those methylated in the first methylase reaction. In some embodiments, the first methylase reaction is carried out with a methylase and a labeled SAM derivative (e.g., aziridine). The nucleic acid molecule may then analyzed for the presence of the labeled aziridine derivative, after which it can be demethylated and re-exposed to one or more methylases and a SAM derivative having a different label. In this way, the in vivo methylated sites can be distinguished from the unmethylated sites. In other embodiments it is not necessary to demethylate prior to using the combination of SAM and a methylase. For instance when using a methylase that does not overlap the normal CpG methylation found in the human genome, for example, then you do not need to demethylate at all. For example, the Bam H1 methylate recognizes the sequence GGATCC and will not be affected by a demethylation step.

It is to be understood that any combination of methods is embraced by the invention, and one of ordinary skill in the art will readily understand which combinations are best suited for a particular application.

In one embodiment, the nucleic acid molecule may be harvested and labeled with one or more sequence specific methylases and SAM labeled derivatives. All “available” methylation sites (i.e., sites that can be methylated but that are not) can be labeled in this way. This labeling pattern can then be compared to a normal methylation pattern (that has all methylation sites labeled). By subtracting the labeling pattern from the normal methylation pattern, it should be possible to identify those methylation sites that were methylated in the nucleic acid molecule at the time of harvest. These methylation patterns may also be compared to a genomic map in order to orient the nucleic acid molecule and the methylation sites, relative to genome.

The method of the invention are able to detect not only the total amount of methylation, but also determine the location and type of methylation.

In some embodiments, particularly those in which all methylation sites are to be determined, regardless of their type or recognition sequence, a plurality of methylases can be used with only one type of SAM derivative (i.e., the same SAM labeled derivative is used by all methylases and thus all methylation sites are labeled with the same label).

It should be clear that the enzymatic methylation reactions may also use SAM as a substrate, thereby transferring a methyl group to the nucleic acid molecule. In this case, the methylated nucleotide can be visualized by labeling the methyl group with tritium, exposing the nucleic acid molecule to a MBP, or a methylation-specific antibodies or antibody fragments, as described below.

The methods that involve direct labeling of methylated nucleic acid molecules that are already methylated make use of agents that recognize and bind to methylated sites on a nucleic acid molecule. Examples of such agents include MBPs and methylation-specific antibodies or antibody fragments.

Several MBPs have been identified in certain disorders including RETT syndrome and gliomas. In particular, RETT syndrome mutations have been mapped to X-linked methyl CpG binding protein 2 (MeCP2). (Amir et al. 1999, 23(2): 185-8.) MeCP2 binds to CpG dinucleotides and thereby represses transcription. Other MBPs that have been identified include MBD1, MBD2, MBD3 and MBD4/MED1. (Schlegel et al. Oncol. Rep. 2002, 9(2):393-5.) Entire MBPs or simply their methylated nucleic acid binding domain can be used to label methylated nucleotides directly. Many MBPs recognize methylated cytosines in the context of a CG dinucleotide, however, the invention intends to embrace MBPs regardless of their binding specificity.

In some aspects of the invention, the methods use methylation-specific antibodies or antibody fragments. It is to be understood that any reference to antibodies applies to antibody fragments equally. These antibodies can have specificity for a number of methylated nucleotides including methylated adenine, methylated thymine, methylated cytosine, methylated guanine, and methylated uridine. Example of methylated nucleotides include N⁶-methyladenine, 4-methylcytosine, 5-methylcytosine, 7-methylguanine, O⁴-methylthymine, O⁶-methylguanine, 6-methyladenosine, 2,2,7-trimethylguanine, and the like.

Antibodies specific for methylated nucleotides can be commercially purchased from Megabase Research Products. Alternatively, unlabeled antibodies of similar specificity have been described by Erlanger and Beiser (PNAS, 52:68, 1964) and by Sano et al., (Biochemica et Biophysica Acta, 951:157, 1988). PCT patent application WO99/10540 published on Mar. 4, 1999 teaches other methods and resources for preparing antibodies specific for methylated nucleotides. Kawarada et al. also teach synthesis in rabbits of antibodies specific for methylated DNA. (Tohuku J. Exp Med. 1986, 149(2):151-161.) Accordingly, methylation-specific antibodies can be generated to any form of methylated nucleotides. In some embodiments, the antibodies are isolated. In other embodiments, the antibodies are provided as antiserum, or ascites fluid. The antibodies may be monoclonal or polyclonal.

The antibodies or MBPs are contacted with the harvested and isolated nucleic acid and allowed to interact with (e.g., bind to) their targets. In some instances it may be desirable to use multiple antibodies or MBPs, each having a specificity unique from that of other antibodies or MBPs, and each labeled with a label distinct from the other antibodies or MBPs. The exposure to different antibodies or MBPs may occur simultaneously or consecutively. The resultant nucleic acid would then be labeled with multiple antibodies or MBPs each bound to a different type of methylated nucleotide and each having a distinct label.

In some instances, the method targets only a particular methylated nucleotide and so includes only antibodies that recognize and bind to a particular type of methylated nucleotide (e.g., 4-methylcytosine but not 5-methylcytosine). Alternatively, the method is indiscriminate and uses a panel of antibodies to a range of methylated nucleotides.

As used herein, the methylation-specific antibodies or antibody fragments and the MBPs bind to the nucleic acid molecules at methylated sites. Therefore, the “binding pattern” of these agents is a surrogate for the “methylation pattern” of the nucleic acid molecule.

Direct binding methods can be used in a number of ways. For example, the binding pattern of the methylation-specific antibodies or antibody fragments or MBPs on harvested nucleic acids can be determined and then compared to a normal methylation pattern. Alternatively, the binding pattern can be compared to a genomic map, such as those available from genome sequencing projects. This identifies sites that are methylated upon harvest of the nucleic acid molecule. In another example, the nucleic acids can be further processed by demethylation, followed by re-methylation with a plurality of methylases and SAM, followed by re-binding of methylation-specific antibodies or antibody fragments or MBPs. In this embodiment, the only labeling of the nucleic acid molecule derives from the antibody or MBP binding and not from the methylase or the SAM.

In still another embodiment, the nucleic acids can be exposed to methylases and SAM labeled derivatives, and then exposed to methylation-specific antibodies having labels different from those of the SAM derivative. In this way, the pre-existing methylated sites can be distinguished from the non-methylated sites in the nucleic acid molecule.

The invention intends to embrace the use of the enzymatic and/or binding methods provided herein, and is not meant to be limited to those examples and combinations recited herein.

In other embodiments, the methylated nucleotide is detected using a methylation-specific antibody fragment. As is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions of the antibody, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. An isolated F(ab′)₂ fragment is referred to as a bivalent monoclonal fragment because of its two antigen binding sites. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd (heavy chain variable region). The Fd fragments are the major determinant of antibody specificity (a single Fd fragment may be associated with up to ten different light chains without altering antibody specificity) and Fd fragments retain epitope-binding ability in isolation.

The terms Fab, Fc, pFc′, F(ab′)₂ and Fv are employed with standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)].

In other aspects, the antibodies may be chimeric, having regions from human antibodies and regions from non human antibodies. The methods for creating such antibodies are known in the art.

In a further embodiment of the method, the nucleic acid molecule may be further processed prior to or following enzymatic methylation, or direct labeling with MBPs or antibodies (or fragments thereof).

For example, the nucleic acid molecule may be treated with a demethylating agent (such as a demethylating enzyme i.e., a demethylase) so as to remove most if not all methyl groups from the nucleic acid molecule. The demethylating agent is used in an amount effective to remove the majority of methyl groups, if not all methyl groups from the nucleic acid molecule. Removal of the majority of methyl groups preferably means greater than 70%, more preferably greater than 80%, even more preferably greater than 90% and most preferably greater than 95% (i.e., 96%, 97%, 98%, 99% and 100%) of methyl groups are removed following exposure to the demethylating enzyme. In some instances, a demethylating agent is used to remove methyl groups from particular methylated nucleotides such as removing a methyl group from methylcytosine. In other embodiments, the demethylating agent indiscriminately removes methyl groups from all methylated nucleotides. In still other embodiments, a set of demethylating agents are used to demethylate the nucleic acid molecule, either consecutively or simultaneously.

In important embodiments, the demethylating agent is a demethylase. A DNA demethylase (DNA dMTase) has been described in PCT patent application WO99/24583, published on May 20, 1999.

Demethylation can be performed prior to, following, or in between methylation analyses. In some instances, it may be preferred that demethylation occur prior to methylation analysis in order to remove all pre-existing methylation which would obscure readout of all potential methylation sites. This may be preferable if the nucleic acid molecule is being sequenced or mapped rather than analyzed for its methylation status. If the methylation status of a nucleic acid molecule is sought instead, then it may be preferable to first perform a methylation analysis, then demethylate (to remove methylation), and then re-methylate in order to identify all potential methylation sites. A comparison of the first and second methylation patterns will provide information about the location, number and frequency of methylated nucleotides in the nucleic acid molecule.

Thus, in some embodiments, following demethylation, the nucleic acid molecule is exposed to one or more sequence-specific methylases in the presence of SAM or a SAM derivative, so that the nucleic acid molecule can be re-methylated or re-labeled, as the case may be. In important embodiments, all known methylases are added to the reaction in order to achieve maximal methylation or labeling of the nucleic acid molecule. If the methylases are used with SAM, the nucleic acid molecules are methylated, and the methylated nucleotides can be visualized using the MBPs and antibodies described herein. If instead the methylases are used with SAM derivatives such as aziridines, the nucleic acid molecules are labeled rather than methylated, and the labels can be detected without the need for MBPs or methylation specific antibodies. As mentioned herein, when SAM derivatives such as aziridine derivatives are used, the label can be on the SAM derivative or the methylase.

The invention also provides methods for mapping sequences (corresponding to methylase recognition sequences) along a nucleic acid molecule. It is possible to obtain positional information for each of these sequences, either relative to each other, or relative to other genomic markers, such as other genomic maps. The sequencing information obtained using these methods is partial, as the method is limited to detecting the recognition sequences of the methylases. Thus, a continuous sequence of the nucleic acid molecule is generally not obtained, but rather the result is a map of the methylase recognition sites distributed along the nucleic acid molecule, and potentially the genome.

Since methylases methylate nucleic acids at particular nucleotides within or near their characteristic recognition sequences, a methylated nucleotide is an indicator of the presence of the recognition sequence. Similarly, the presence of a labeled nucleotide following exposure to a methylase and a SAM labeled derivative is also an indicator of the presence of the recognition sequence.

MBPs and methylation-specific antibodies (or fragments thereof) can also be used for sequencing purposes, particularly if these are used to detect methylation resulting from a controlled in vitro methylation reaction, such as a sequence specific methylation. For example, a nucleic acid molecule may be methylated using a sequence-specific methylase and SAM, following which it can be exposed to an antibody or a MBPs specific for the type of methylated nucleotide that is known to be generated from the methylation reaction. The binding of the antibody or the MBP indicates that a particular methylated nucleotide is present, and this in turn indicates that a particular known recognition sequence exists at or near the methylated nucleotide. The methylation reactions and labelings may be conducted in consecutive order, particularly if the methylated nucleotide that is generated is the same. If the methylases generate different methylated nucleotides, then they may be incubated together, as could the antibodies or MBPs that recognize the methylated nucleotides. MBPs and methylation-specific antibodies can also be used consecutively.

The following is a brief description of how sequence information can be obtained from a nucleic acid molecule. Nucleic acid molecules harvested and isolated from a biological sample (such as a tissue sample or a bodily fluid or an ex vivo tissue culture) is first exposed to a demethylating enzyme such as that described above. The exposure is continued until preferably a majority of the methylated nucleotides are demethylated. Following demethylation, the nucleic acid molecule is sequentially exposed to pre-determined methylase and labeled SAM derivative combinations. In an important embodiment, as many methylases as possible are used in a sequential fashion. Each methylase should have a unique and distinct recognition sequence such that labeling of the nucleic acid molecule with the particular SAM derivative (with which it is paired) is indicative of the recognition sequence. The result is that the nucleic acid molecule will be labeled with a number of different labels, each corresponding to a particular, known recognition sequence. Both strands of the nucleic acid can be labeled using this technique, and both strands can be analyzed either together or individually.

As an example, two or more methylases can be used sequentially to attach, for example, different fluorophores to a nucleic acid molecule. Each fluorophore corresponds to the presence of a different recognition sequence. Thus, a nucleic acid molecule can be labeled with one methylase/fluorophore combination followed by another one, and so on. As a further example, EcoR1, BamH1, and PVU II methylases, each identify a 6 base recognition sequence. Individually, each unique 6 base sequence will occur, on average, 1:4096 base pairs. Therefore, if all three methylases are used, each with a unique SAM derivative, then it should be possible to obtain a sequence map of the nucleic acid molecule at a resolution at least on the order of 1364 base pairs. The more methylases that are used, the higher the resolution. In some embodiments, the resolution is limited by the resolution limit of the system being used. In some embodiments, the resolution is at least 100 bp, at least 200 bp, at least 300 bp, at least 400 bp, at least 500 bp, at least 600 bp, at least 700 bp, at least 800 bp, or more. The resolution may also be limited by the ability of methylases to recognize, bind and/or act on sites that are already methylated. Thus, if several methylases are used, recognition sequences that overlap may not all be detected.

Each nucleic acid molecule so labeled will have a unique pattern of methylation recognition sites. This unique pattern can be akin to a “fingerprint” of the nucleic acid molecule. The greater the number of different methylases used (each with a distinct recognition sequence), the more sequence information is available.

In some embodiments, the sequencing information can be compared to genomic sequencing information that is available from sources such as the human genome project. The methylation patterns deduced using the methods of the invention can also be superimposed onto physical genomic maps. These maps (including sequence, motif and structural maps) are available from public sources such as the human genome project, or the genome sequencing projects of other organisms. Superimposition of the methylation patterns derived using the methods of the invention helps to locate the region of the genome that is being analyzed. The physical maps of genomes are therefore used as references for orienting the methylation patterns determined using the methods of the invention. Moreover, it also helps to identify the genetic loci that are methylated, such as active or silent genetic loci, imprinted loci, as well as previously unknown loci, or loci to which no function has yet been ascribed. All aspects of the invention can include the step of comparing the methylation pattern to a physical map of the genome or part thereof for that particular species. In some embodiments, a sample of nucleic acids is divided into two equal aliquots, and each aliquot is processed differently. One aliquot may be demethylated, while the other is labeled with the MBPs or methylation specific antibodies of the invention. The demethylated aliquot can then be re-methylated in order to methylate all possible sites. Both aliquots can be processed with preferably an independent marker that can be used to align the nucleic acid molecules of the two aliquots relative to each other. This independent marker may simply be a nucleic acid probe to a short nucleotide sequence. This probe would be distinctly labeled from the other labels used in the methyl labeling reactions. The aliquots are then analyzed and the positional data is subtracted from the other, using the pattern of the third marker for alignment. This subtraction should result in differences that represent nucleotides not methylated at the time of harvest. Those of skill in the art will understand how to manipulate the order of these reactions in order to derive differences that correspond to those nucleotides methylated at the time of harvest.

The methods of the invention involve the use of agents that are labeled. These agents include methylases, SAM derivatives, antibodies, antibody fragments, and MBPs. As used herein, these agents are bound, preferably covalently to a detectable label. A detectable label includes a label that is directly detectable and a label that is indirectly detectable. Generally, detection of the label involves an absorption or an emission of energy by the label. The label can be detected directly by its ability to emit and/or absorb light of a particular wavelength. A label can be detected indirectly by its ability to bind, recruit and, in some cases, cleave another moiety which itself may emit or absorb light of a particular wavelength. An example of indirect detection is the use of a first enzyme label which cleaves a substrate into visible products. The label may also be an enzyme substrate.

The label may be of a chemical, carbohydrate, lipid, peptide or nucleic acid nature although it is not so limited. Other detectable labels include radioactive isotopes such as P³² or H³, chemiluminescent substrates, chromogenic substrates, luminescent markers such as fluorochromes, such as fluorescein isothiocyanate (FITC), TRITC, rhodamine, tetramethylrhodamine, R-phycoerythrin, Cy-3, Cy-5, Cy-7, Texas Red, Phar-Red, allophycocyanin (APC), etc., optical or electron density markers, etc., biotin, avidin, digoxigenin, or epitope tags such as the FLAG epitope or the HA epitope, biotin, avidin and enzyme tags such as alkaline phosphatase, horseradish peroxidase, P-galactosidase, etc.

Also envisioned by the invention is the use of semiconductor nanocrystals such as quantum dots (i.e., Qdots), described in U.S. Pat. No. 6,207,392 as labels. Qdots are commercially available from Quantum Dot Corporation. The label may also be an electrically charged transducing molecule, a nuclear magnetic resonance molecule, a semiconductor nanocrystal, an electromagnetic molecule, etc.

In still other embodiments, the detectable label is a ligand or a receptor of a ligand/receptor pair, a microbead, a magnetic bead, or an affinity molecule.

Linkage of labels to the agents of the invention can be carried out by a number of known covalent and non-covalent processes. These linkages are routine in the art. A universal linkage system that can be used to link a variety of labels to a variety of agents is described by van Gijlswijk et al. (Expert Rev Mol Diagn 2001, 1(1):81-91.)

The detectable labels can also be antibodies or antibody fragments and their corresponding antigen or hapten binding partners. (These antibodies and fragments thereof are not to be confused with the methylation-specific antibodies and fragments discussed herein.) Detection of such bound antibodies and proteins or peptides is accomplished by techniques known to those skilled in the art. Use of hapten conjugates such as digoxigenin or dinitrophenyl is also well suited herein. Antibody/antigen complexes which form in response to hapten conjugates are easily detected by linking a label to the hapten or to antibodies which recognize the hapten and then observing the site of the label. Alternatively, the antibodies can be visualized using secondary antibodies or fragments thereof that are specific for the primary antibody used. Polyclonal and monoclonal antibodies may be used. Antibody fragments include Fab, F(ab)₂, Fd and antibody fragments which include a CDR3 region.

The label emits a signal and this signal must be detected by a detection system. The detection system can be selected based on the nature of the label, and can be selected from the group of detection systems consisting of a fluorescent detection system, an electrical detection system, a photographic film detection system, a chemiluminescent detection system, an enzyme detection system, an atom force microscopy (AFM) detection system, a scanning tunneling microscopy (STM) detection system, an optical detection system, a nuclear magnetic resonance (NMR) detection system, a near field detection system, total internal reflection (TIR) system, and an electromagnetic detection system, but is not so limited.

The invention intends that any combination of labels can be used along the length of a nucleic acid. This means that a nucleic acid molecule may be labeled with a fluorophore, a chromophore, a nuclear magnetic resonance label and a semiconductor nanocrystal along its length and it may be so analyzed by the systems described herein. These systems have the capability of detecting signals from a number of different “signal modalities.”

Analysis of the nucleic acid involves detecting signals from the labels, and determining the relative position of those labels relative to one another. In some instances, it may be desirable to further label the nucleic acid molecule with a standard marker that facilitates comparing the information so obtained with that from other nucleic acids analyzed. For example, the standard marker may be a backbone label, or a label that binds to a particular sequence of nucleotides (be it a unique sequence or not), or a label that binds to a particular location in the nucleic acid molecule (e.g., an origin of replication, a transcriptional promoter, a centromere, etc.).

One subset of backbone labels are nucleic acid stains that bind nucleic acids in a sequence independent manner. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS75 1, and hydroxystilbamidine. All of the aforementioned nucleic acid stains are commercially available from suppliers such as Molecular Probes, Inc. Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red).

Unlike prior art methods that analyzed total methyl content in a nucleic acid molecule, the approaches described herein are able to determine total methyl content in a nucleic acid molecule, and also to locate the position and type of methylation. The methods do not require amplification, restriction endonuclease digestion or other processing of nucleic acid molecule, as required by the prior art methods.

The nucleic acid molecules are analyzed using linear polymer analysis systems. A linear polymer analysis system is a system that analyzes polymers in a linear manner (i.e., starting at one location on the polymer and then proceeding linearly in either direction therefrom). As a polymer is analyzed, the detectable labels attached to it are detected in either a sequential or simultaneous manner. When detected simultaneously, the signals usually form an image of the polymer, from which distances between labels can be determined. When detected sequentially, the signals are viewed in histogram (signal intensity vs. time), that can then be translated into a map, with knowledge of the velocity of the nucleic acid molecule. It is to be understood that in some embodiments, the nucleic acid molecule is attached to a solid support, while in others it is free flowing. In either case, the velocity of the nucleic acid molecule as it moves past, for example, an interaction station or a detector, will aid in determining the position of the labels, relative to each other and relative to other detectable markers that may be present on the nucleic acid molecule.

Accordingly, the linear polymer analysis systems are able to deduce not only the total amount of label on a nucleic acid molecule, but perhaps more importantly, the location of such labels. The ability to locate and position the labels (and thus the methylation sites) allows the methylation patterns to be superimposed on other genetic maps, in order to identify the regions of the genome that are affected. In preferred embodiments, the linear polymer analysis systems are capable of analyzing nucleic acid molecules individually (i.e., they are single molecule detection systems).

An example of such a system is the Gene Engine™ system described in PCT patent applications WO98/35012 and WO00/09757, published on Aug. 13, 1998, and Feb. 24, 2000, respectively, and in issued U.S. Pat. No. 6,355,420 Bi, issued Mar. 12, 2002. The contents of these applications and patent, as well as those of other applications and patents, and references cited herein are incorporated by reference in their entirety. This system allows single nucleic acid molecules to be passed through an interaction station in a linear manner, whereby the nucleotides in the nucleic acid molecules are interrogated individually in order to determine whether there is a detectable label conjugated to the nucleic acid molecule. Interrogation involves exposing the nucleic acid molecule to an energy source such as optical radiation of a set wavelength. In response to the energy source exposure, the detectable label on the nucleotide (if one is present) emits a detectable signal. The mechanism for signal emission and detection will depend on the type of label sought to be detected.

Other single molecule nucleic acid analytical methods which involve elongation of DNA molecule can also be used in the methods of the invention. These include optical mapping (Schwartz et al., 1993, Science 262:110-113; Meng et al., 1995, Nature Genet. 9:432; Jing et al., Proc. Natl. Acad. Sci. USA 95:8046-8051) and fiber-fluorescence in situ hybridization (fiber-FISH) (Bensimon et al., Science 265:2096; Michalet et al., 1997, Science 277:1518). In optical mapping, nucleic acid molecules are elongated in a fluid sample and fixed in the elongated conformation in a gel or on a surface. Restriction digestions are then performed on the elongated and fixed nucleic acid molecules. Ordered restriction maps are then generated by determining the size of the restriction fragments. In fiber-FISH, nucleic acid molecules are elongated and fixed on a surface by molecular combing. Hybridization with fluorescently labeled probe sequences allows determination of sequence landmarks on the nucleic acid molecules. Both methods require fixation of elongated molecules so that molecular lengths and/or distances between markers can be measured. Pulse field gel electrophoresis can also be used to analyze the labeled nucleic acid molecules. Pulse field gel electrophoresis is described by Schwartz et al. in Cell, 1984, 37:67. Other nucleic acid analysis systems are described by Otobe et al. (NAR, 2001, 29:109), Bensimon et al. in U.S. Pat. No. 6,248,537, issued Jun. 19, 2001, Herrick and Bensimon (Chromosome Res 1999, 7(6):409-423), Schwartz in U.S. Pat. No. 6,150,089 issued Nov. 21, 2000 and U.S. Pat. No. 6,294,136, issued Sep. 25, 2001. Other linear polymer analysis systems can also be used, and the invention is not intended to be limited to solely those listed herein.

In some aspects, therefore, the methylation patterns, including level, location, and type of methylation, deduced are compared to “normal” methylation patterns. Comparison of the methylation patterns deduced using the methods of the invention to normal methylation patterns can also provide insight into the biological relevance of particular methylation patterns. “Normal” methylation patterns can be the methylation patterns of nucleic acid molecules from normal subjects, or from normal nucleic acid samples from nucleic acid depositories. Preferably, the normal methylation pattern is derived from apparently healthy subjects who have no prior history of methylation-mediated disorders. More preferably, the normal methylation pattern is that pattern in a tissue of a normal subject corresponding to the tissue sampled for the test subject. As an example, breast tumors are, in some cases, sufficiently delineated to the extent that such tissue can be distinguished from the surrounding normal breast tissue. This delineation facilitates selective removal of diseased breast tissue, such as occurs in non-radical mastectomies (e.g., lumpectomy). Similarly, such delineation can be used in the present invention to harvest both suspected diseased tissue and normal tissue from a given subject.

A “normal” level of methylation can also be a range, for example, where a population is used to obtain a baseline range for a particular group into which the subject falls. Thus, the “normal” value can depend upon a particular population selected. Such normal levels can be established as pre-selected values, taking into account the category in which an individual falls. Appropriate ranges and categories can be selected with no more than routine experimentation by those of ordinary skill in the art. Either the mean or another pre-selected number within the range can be established as the normal pre-selected value.

Accordingly, in some aspects, it will be generally be necessary to perform a methylation analysis on the normal cell or the normal tissue as well as on the biological sample. Alternatively, the normal methylation pattern may be pre-determined and stored as data, that is accessible and comparable to the methylation patterns determined herein. In some instances, the comparison will demonstrate that the nucleic acid molecule is hypomethylated, while in other instances, the comparison will demonstrate that the nucleic acid molecule is hypermethylated. The method will yield information regarding not only the total level of methylation within the nucleic acid molecule but also the position along the nucleic acid molecule at which methylation occurs. This is an advantage over some prior art methods which provide information regarding total methylation but do not provide information regarding methylation position.

The methods can be used to identify methylation patterns that are disorder-specific. As an example, the cells may be cancerous and their methylation status in vivo may be analyzed in order to determine whether particular regions or nucleotides of the nucleic acid molecule are preferentially methylated in the cancerous cells. Further analysis can involve performing the same analysis on nucleic acid molecules harvested from a normal, non-cancerous cell, and then comparing the pattern of methylation between the cancerous and non-cancerous samples. This comparison can lead to the identification of patterns of methylation that are associated with cancer, or a particular cancer type, or with a susceptibility to cancer or a particular type of cancer.

A genetic locus that is hypomethylated refers to a region of a nucleic acid molecule that contains fewer methylated nucleotides than a control nucleic acid molecule from a normal cell. A genetic locus that is hypermethylated refers to a region of a nucleic acid molecule that contains more methylated nucleotides than a control nucleic acid molecule from a normal cell. A normal cell as used herein intends that the cell is not diseased, and that does not have an above-normal risk of being diseased at some later time. Both hypo- and hyper-methylation have been associated with disorders, the finding of hypo- or hyper-methylation can be instructive with respect to particular disorders. Hypermethylation has been observed in cancers, particularly at the promoter regions of genetic loci, where it is believed to cause silencing of gene expression from the locus. (Wistuba et al. Semin. Oncol. 2201, 28(2 Supp 4):3-13.) Thus, comparison of methylation patterns from subjects having a cancer with normal methylation patterns can lead to the identification of genetic loci that are either hypomethylated or hypermethylated as a result of a particular disorder or susceptibility to a particular disorder.

The identification of methylation “hot spots” in a nucleic acid molecule can also be performed using the methods of the invention. Methylation hot spots are regions of a nucleic acid molecule having an above-normal level of methylated nucleotides (i.e., regions of concentrated methylation). These hot spots can be used to identify genetic loci that are important in the development of a disorder, and can then be used as markers of the disorder or of the risk of developing the disorder. Methylation hot spots can also correspond to known genetic loci, such as loci known to be hypermethylated in particular disorders. For example, the promoter regions in both the retinoblastoma and the Wilms Tumor genes are reportedly hypermethylated in some tumors. Identification of methylation hot spots can be used as a first screen for typing of a biological sample suspected of being malignant.

The invention also provides methods for identifying subjects having or at risk of developing a disorder characterized by abnormal methylation. Abnormal methylation generally can refer to methylation levels that are lower than, or greater than, the level of methylation in normal cells (i.e., normal methylation). Abnormal methylation also includes a methylation pattern that is different from a normal methylation pattern. These latter differences can be a difference in location or type of methylation between sample and normal methylation patterns.

As used herein, a subject includes a human and a non-human subject. In some preferred embodiments, the subject is a mammal. Subjects include but are not limited to humans, primates, domesticated animals such as dogs and cats, agricultural livestock such as cows, pigs, horses, chickens, sheep, etc., aquaculture species such as fish and shellfish, zoo animals such as bears, lions, etc., laboratory animals such as mice, rats, rabbits, etc. and the like.

Methylation disorders including but are not limited to cancer, Beckwith-Wiedemann syndrome, familial Prader-Willi syndrome, ICF immunodeficiency syndrome, X chromosome inactivation associated conditions such as fragile X syndrome, and disorders involving inappropriate parental imprinting.

In some embodiments, the disorder is a proliferative disorder such as cancer. A cancer is defined as an uncontrolled, abnormal growth of cells, which can either remain localized or may disseminate throughout the body via the bloodstream or the lymphatic system, and thereby seed a secondary site (i.e., a metastasis). Diagnosis as used herein is directed to a cancer at its primary site and/or at a metastatic site. Examples of cancers that can be diagnosed include: biliary tract cancer; brain cancer, including glioblastomas and medulloblastomas; breast cancer; cervical cancer; choriocarcinoma; colon cancer; endometrial cancer; esophageal cancer; gastric cancer; hematological neoplasms, including acute lymphocytic and myelogenous leukemia; chronic lymphocytic and myelogenous leukemia, multiple myeloma; AIDS associated leukemias and adult T-cell leukemia lymphoma; intraepithelial neoplasms, including Bowen's disease and Paget's disease; liver cancer; lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreas cancer; prostate cancer; colorectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma teratomas and choriocarcinomas), stromal tumors and germ cell tumors; thyroid cancer, including thyroid adenocarcinoma and medullar carcinoma; and renal cancer including adenocarcinoma and Wilms' tumor. Preferably, the invention seeks to diagnose disorders (or susceptibility thereto) such a breast cancer, cervical cancer, leukemia, ovarian cancer and prostate cancer.

The methylation status of a nucleic acid molecule can be related to the conditions under which the cells from which the nucleic acid molecule is derived were subjected to prior to nucleic acid harvest. For example, the cells may have been exposed to a compound (e.g., an anti-cancer agent or an inhibitor of methyltransferase) either in vivo or ex vivo. Thus, the methods of the invention are useful in determining the effect of such compounds on the nucleic acid molecule, and potentially in turn on the cell, tissue or organism.

Another application of the methods described herein is the assessment of the efficacy of therapeutic treatments. In some instances, the therapeutic efficacy is determined by a decrease, or an increase in the level of methylation, or a change in the location or type of methylation, or a total absence of a particular methylation pattern. In an important embodiment, the therapeutic treatment is the administration of an anti-cancer agent. In other embodiments, the therapeutic treatment is a demethylating agent. A demethylating agent is an agent which directly or indirectly causes a reduction in the level of methylation of a nucleic acid molecule. Demethylating agents include inhibitors of methylating enzymes such as methyltransferases. Examples of demethylating agents useful in the invention include 5-azacytidine, 5-aza-2′deoxycytidine (also known as Decitabine in Europe), 5,6-dihydro-5-azacytidine, 5,6-dihydro-5-aza-2′deoxycytidine, 5-fluorocytidine, 5-fluoro-2′deoxycytidine, and short oligonucleotides containing 5-aza-2′deoxycytosine, 5, 6-dihydro-5-aza-2′deoxycytosine, and 5-fluoro -2′deoxycytosine. All of the foregoing agents act as DNA methyltransferase inhibitors. Agents like these, such as the derivatives mentioned, are most effective if capable of being incorporated into a nucleic acid, preferably, DNA. Other agents reported to inhibit DNA methyltransferases and/or cause demethylation in vitro include procanamide and S-adenosyl homocysteine. Several candidate small molecule demethylating agents, including inhibitors of methyltransferase, which do not require nucleic acid incorporation to manifest their effects, are currently being developed.

The invention provides a method whereby a sample can be harvested from a subject either diagnosed a particular disorder (such as for example cancer) or a subject at risk of developing such a disorder. The sample may be a tissue, a cell population or a bodily fluid, and would usually be acquired from a biopsy from the subject. Nucleic acid molecules from the sample are harvested and isolated and analyzed to determine their methylation status, according to the methods of the invention. A “pre-treatment” methylation profile or pattern of one, more than one or all nucleic acid molecules can be so determined. The subject would then be treated with the therapeutic treatment and following such treatment, another biological sample would be harvested from the subject. Nucleic acid molecules are harvested and isolated from the “post-treatment” sample, and analyzed to determine their methylation status. Preferably, the samples are harvested from the same tissue, region of the body, or bodily fluid. For example, if the subject has a tumor, both the pre-treatment and post-treatment samples would derived from the tumor. Generally, the samples will also be taken from those cells, tissues, or fluids thought to be affected by the disorder. In other instances however it may be desirable to investigate the effect of the therapeutic treatment on non-diseased cells or tissues. For example, it may be desirable to determine the specificity of particular therapeutic treatments in order to identify treatments that more specifically target diseased cells or tissues while leaving normal cells or tissues intact.

Other anti-cancer agents that can be tested using the methods of the invention include those listed below. It is to be understood that these compounds can be tested using the methods of the invention to the extent that they lead to a change in methylation status of analyzed nucleic acid molecules.

Other anti-cancer agents include DNA damaging anti-cancer agents such as topoisomerase inhibitors (e.g., etoposide, ramptothecin, topotecan, teniposide, mitoxantrone), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), and radiation therapy.

Other anti-cancer agents include immunotherapeutic agents such as Ributaxin, Herceptin (trastuzumab), Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab, ImmuRAIT-CEA, immunostimulant peptides, oligonucleotides,.

Other anti-cancer agents include cancer vaccines such as EGF, anti-idiotypic cancer vaccines, Gp75 antigen, GMK melanoma vaccine, MGV ganglioside conjugate vaccine, Her2/neu, Ovarex, M-Vax, O-Vax, L-Vax, STn-KHL theratope, BLP25 (MUC-1), liposomal idiotypic vaccine, Melacine, peptide antigen vaccines, toxin/antigen vaccines, MVA-based vaccine, PACIS, BCG vaccine, TA-HPV, TA-CIN, DISC-virus and ImmuCyst/TheraCys.

Other anti-cancer agents include biological response modifiers such as cytokines such as interferon, interleukins and lymphokines (e.g., IL-2), interferon agonists, hemopoietic growth factors (e.g., erythropoietin, GM-CSF, G-CSF), bFGF inhibitor, insulin-like growth factor-1 receptor inhibitor.

Other anti-cancer agents include hormone therapy such as adrenocorticosteriods (e.g., prednisone, methylprednisolone, dexamethasone), androgens (e.g., fluoxymesterone), anti-androgens (e.g., flutamide), estrogens (e.g., diethylstilbestrol, ethinyl estradiol), anti-estrogens (e.g., tamoxifen), progestins (e.g., medroxyprogesterone, megestrol acetate), aromatase (aminoglutethimide), gonadotropin-releasing hormone agonists (e.g., leuprolide), somatostatin analogues (e.g., octreotide).

Other anti-cancer agents include angiogenesis inhibitors such as basic FGF, VEGF, angiopoietins, angiostatin, endostatin, TNFα, TNP-470, thrombospondin-1, platelet factor 4, CAI, and certain members of the integrin family of proteins.

Other anti-cancer agents include Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Iproplatin; Irinotecan Hydrochloride; Lanreotide Acetate; Letrozole; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Taxotere; Temoporfin; Teniposide; Teroxirone; Testolactone; Thiamiprine; Thioguanine; Thiotepa; Tiazofurin; Tirapazamine; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other anti-cancer agents include methotrexate, vincristine, adriamycin, cisplatin, nonsugar containing chloroethylnitrosoureas, mitomycin C, dacarbazine, fragyline, Meglamine GLA, valrubicin, carnustaine and poliferposan, MM1270, BAY 12-9566, RAS famesyl transferase inhibitor, famesyl transferase inhibitor, MMP, MTA/LY231514, LY264618/Lometexol, Glamolec, CI-994, TNP-470, PKC412, Valspodar/PSC833, Novantrone/Mitroxantrone, Metaret/Suramin, Batimastat, E7070, BCH-4556, CS-682, 9-AC, AG3340, AG3433, IncelIVX710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Xeload/Capecitabine, Furtulon/Doxifluridine, Oral Taxoid, SPU-077/Cisplatin, HMR 1275/Flavopiridol, CP-358 (774)/EGFR, CP-609 (754)/RAS oncogene inhibitor, BMS182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Doxil/liposomal doxorubicin, Caelyx/liposomal doxorubicin, Fludara/Fludarabine, Pharmarubicin/Epirubicin, DepoCyt, ZD 1839, LU 79553/Bis-Naphtalimide, LU 103793/Dolastain, Caetyx/liposomal doxorubicin, Gemzar/Gemcitabine, ZD 0473/Anormed, YM 116, lodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, nitrosoureas, alkylating agents such as melphelan, cyclophosphamide, Aminoglutethimide, Asparaginase, Busulfan, Carboplatin, Chlorombucil, Cytarabine HCI, Dactinomycin, Daunorubicin HCl, Estramustine phosphate sodium, Floxuridine, Flutamide, Hydroxyurea (hydroxycarbamide), Ifosfamide, Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Thioguanine, Thiotepa, Vinblastine sulfate, Amsacrine (m-AMSA), Azacitidine, Hexamethylmelamine (HMM), Mitoguazone (methyl-GAG; methyl glyoxal bisguanylhydrazone; MGBG), Pentostatin (2′deoxycoformycin), Semustine (methyl-CCNU), Teniposide (VM-26) and Vindesine sulfate.

Other anti-cancer agents include 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amasacrine; anagrelide; anastrozole; andrographolide; antagonist D; antagonist G; antarelix; anti-dorsalizing morphogenetic protein-1; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factorsaporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti cancer compound; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O⁶-benzylguanine; octreotide; okicenone; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Other anti-cancer agents include anti-cancer supplementary potentiating agents. Examples include Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and multiple drug resistance reducing compounds such as Cremaphor EL.

Other compounds that are useful in combination with anti-cancer agents include Piritrexim Isethionate; the antipro static hypertrophy compound, Sitogluside; the benign pro static hyperplasia therapy compound, Tamsulosin Hydrochloride; the prostate growth inhibitor, Pentomone; radioactive compounds such as Fibrinogen 1 125, Fludeoxyglucose F 18, Fluorodopa F 18, Insulin I 125, Insulin I 131, Iobenguane I 123, Iodipamide Sodium I 131, Iodoantipyrine I 131, Iodocholesterol I 131, Iodohippurate Sodium I 123, Iodohippurate Sodium I 125, Iodohippurate Sodium I 131, Iodopyracet I 125, Iodopyracet I 131, Iofetamine Hydrochloride I 123, Iomethin I 125, Iomethin I 131, Iothalamate Sodium I 125, Iothalamate Sodium I 131, Iotyrosine I 131, Liothyronine I 125, Liothyronine I 131, Merisoprol Acetate Hg 197, Merisoprol Acetate Hg 203, Merisoprol Hg 197, Selenomethionine Se 75, Technetium Tc 99m Antimony Trisulfide Colloid, Technetium Tc 99m Bicisate, Technetium Tc 99m Disofenin, Technetium Tc 99m Etidronate, Technetium Tc 99m Exametazime, Technetium Tc 99m Furifosmin, Technetium Tc 99m Gluceptate, Technetium Tc 99m Lidofenin, Technetium Tc 99m Mebrofenin, Technetium Tc 99m Medronate, Technetium Tc 99m Medronate Disodium, Technetium Tc 99m Mertiatide, Technetium Tc 99m Oxidronate, Technetium Tc 99m Pentetate, Technetium Tc 99m Pentetate Calcium Trisodium, Technetium Tc 99m Sestamibi, Technetium Tc 99m Siboroxime, Technetium Tc 99m Succimer, Technetium Tc 99m Sulfur Colloid, Technetium Tc 99m Teboroxime, Technetium Tc 99m Tetrofosmin, Technetium Tc 99m Tiatide, Thyroxine I 125, Thyroxine I 131, Tolpovidone I 131, Triolein I 125 and Triolein I 131.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. It is intended to encompass all such modifications and equivalents within the scope of the appended claims.

All references, patents and patent applications that are recited in this application are incorporated by reference herein in their entirety.

We claim:

1. A method for analyzing a nucleic acid molecule, comprising:
exposing a nucleic acid molecule to a sequence-specific methylase and an S-adenosyl methionine labeled derivative,
allowing the sequence-specific methylase to label the nucleic acid molecule with the S-adenosyl methionine labeled derivative, and
determining a labeling pattern in the nucleic acid molecule using a linear polymer analysis system,
wherein the labeling pattern is indicative of a methylation pattern of the nucleic acid molecule.

2. The method of claim 1, wherein the nucleic acid molecule is a non in vitro amplified nucleic acid molecule.

3. The method of claim 1, wherein the nucleic acid molecule is DNA or RNA.

4. The method of claim 3, wherein the DNA is genomic DNA.

5. The method of claim 1, wherein the S-adenosyl methionine labeled derivative is an aziridine derivative.

6. The method of claim 1, wherein the labeling pattern in the nucleic acid molecule is determined using a method selected from the group consisting of Gene Engine™, optical mapping, and DNA combing.

7. The method of claim 1, wherein the nucleic acid molecule is exposed to a demethylating enzyme in an amount effective to demethylate the nucleic acid molecule, prior to exposure to the sequence-specific methylase and the S-adenosyl methionine labeled derivative.

8. The method of claim 1, further comprising, after determining the labeling pattern in the nucleic acid molecule,
exposing the nucleic acid molecule to a demethylating enzyme in an amount effective to demethylate the nucleic acid molecule,
re-exposing the nucleic acid molecule to a sequence-specific methylase and a S-adenosyl methionine labeled derivative,
allowing the sequence-specific methylase to re-label target nucleotides in the nucleic acid molecule with the S-adenosyl methionine labeled derivative, and
determining a labeling pattern in the nucleic acid molecule.

9. The method of claim 8, wherein the labeling patterns prior to exposure to the demethylating enzyme and following the exposure to the demethylating enzyme are compared.

10. The method of claim 1, wherein the nucleic acid molecule is exposed to a station to produce a signal arising from the nucleotide modification, and detecting the signal using a detection system.

11. The method of claim 1, wherein the S-adenosyl methionine labeled derivative comprises a label selected from the group consisting of a fluorescent molecule, a chemiluminescent molecule, a radioisotope, an enzyme substrate, a biotin molecule, an avidin molecule, an electrically charged transducing molecule, a nuclear magnetic resonance molecule, a semiconductor nanocrystal, an electromagnetic molecule, an electrically conducting particle, a ligand, a microbead, a magnetic bead, a Qdot, a chromogenic substrate, an affinity molecule, a protein, a peptide, a nucleic acid, a carbohydrate, an antigen, a hapten, an antibody, an antibody fragment, and a lipid.

12. The method of claim 1, wherein the detection system is selected from the group consisting of a fluorescent detection system, an electrical detection system, a photographic film detection system, a chemiluminescent detection system, an enzyme detection system, an atom force microscopy (AFM) detection system, a scanning tunneling microscopy (STM) detection system, an optical detection system, a nuclear magnetic resonance (NMR) detection system, a near field detection system, a total internal reflection (TIR) system and a electromagnetic detection system.

13. The method of claim 1, further comprising labeling the nucleic acid molecule with a backbone label.

14. The method of claim 1, further comprising comparing the methylation pattern with a normal methylation pattern.

15. The method of claim 14, wherein the normal methylation pattern is determined from a normal subject.

16. The method of claim 14, wherein the normal methylation pattern is determined from a physical genome map.

17. A method for analyzing a nucleic acid molecule, comprising:
exposing the nucleic acid molecule to a methylated nucleic acid binding protein, and
determining the pattern of binding of the methylated nucleic acid binding protein to the nucleic acid molecule using a linear polymer analysis system,
wherein the pattern of binding of the methylated nucleic acid binding protein is indicative of a methylation pattern of the nucleic acid molecule.

18. The method of claim 17, wherein the nucleic acid molecule is DNA or RNA.

19. The method of claim 17, wherein the DNA is genomic DNA.

20. The method of claim 17, wherein the nucleic acid molecule is a non in vitro amplified nucleic acid molecule.

21. The method of claim 17, wherein the linear polymer analysis system is a single molecule detection system.

22. The method of claim 17, wherein the linear polymer analysis system is selected from the group consisting of Gene Engine™, optical mapping, fiber-FISH, and DNA combing.

23. The method of claim 22, wherein the linear polymer analysis system is Gene Engine™.

24. The method of claim 17, wherein the methylated nucleic acid binding protein is selected from the group consisting of MBD1, MBD2, MBD3, MBD4/MED1 and MeCP2.

25. The method of claim 17, wherein the methylated nucleic acid binding protein is labeled with a detectable label.

26. A method for analyzing a single nucleic acid molecule, comprising:
exposing the nucleic acid molecule to a methylation-specific antibody or antibody fragment, and
determining the pattern of binding of the methylation-specific antibody or antibody fragment to the nucleic acid molecule using a linear polymer analysis system,
wherein the pattern of binding of the methylation-specific antibody or antibody fragment is indicative of a methylation pattern of the nucleic acid molecule.

27. The method of claim 26, further comprising comparing the methylation pattern to a normal methylation pattern.

28. The method of claim 26, wherein the normal methylation pattern is determined from a normal subject.

29. The method of claim 26, wherein the methylation-specific antibody or antibody fragment binds specifically to a methylated nucleotide selected from the group consisting of 6methyladenosine, 4-methylcytosine, 5-methylcytosine, O⁶-methylguanine, and O⁴-methylthymine.

30. The method of claim, wherein the methylation-specific antibody or antibody fragment is an antibody.

31. The method of claim 26, further comprising, after determining the pattern of binding of the methylation-specific antibody or antibody fragment,
exposing the nucleic acid molecule to a demethylating enzyme in an amount effective to de-methylate the nucleic acid molecule,
re-exposing the nucleic acid molecule to a sequence-specific methylase and an S-adenosyl methionine labeled derivative,
allowing the sequence-specific methylase to label the nucleic acid molecule with the S-adenosyl methionine labeled derivative, and
determining the labeling pattern in the nucleic acid molecule,
wherein the labeling pattern is indicative of a methylation pattern of the nucleic acid molecule.

32. The method of claim 31, further comprising, comparing the methylation pattern prior to exposure to the demethylating enzyme with the methylation pattern after exposure to the demethylating enzyme.

33. A method for identifying a subject having or at risk for developing a disorder characterized by abnormal methylation of a nucleic acid molecule, comprising:
determining a methylation pattern of a nucleic acid molecule in a biological sample from a subject, and
comparing the methylation pattern of the nucleic acid molecule to a control, wherein a difference in the methylation pattern of the nucleic acid molecule as compared to the control identifies a subject having or at risk of developing a disorder.

34. The method of claim 33, wherein the methylation pattern is determined by
exposing the nucleic acid molecule to a methylation-specific antibody or antibody fragment to the nucleic acid molecule, and
determining the pattern of binding of the methylation-specific antibody or antibody fragment to the nucleic acid molecule using a linear polymer analysis system.

35. The method of claim 34, wherein the methylation-specific antibody or antibody fragment is labeled with a detectable label.

36. The method of claim 33, wherein the methylation pattern is determined by
exposing the nucleic acid molecule to a methylated nucleic acid binding protein, and
determining the pattern of binding of the methylated nucleic acid binding protein to the nucleic acid molecule using a linear polymer analysis system.

37. The method of claim 36, wherein the methylated nucleic acid binding protein is labeled with a detectable label.

38. The method of claim 33, wherein the pattern of methylation is determined by
exposing the nucleic acid molecule to a sequence-specific methylase and an S-adenosyl methionine labeled derivative,
allowing the sequence-specific methylase to label the nucleic acid molecule with the S-adenosyl methionine labeled derivative, and
determining the labeling pattern in the nucleic acid molecule using a linear polymer analysis system.

39. The method of claim 38, wherein the S-adenosyl methionine labeled derivative is an aziridine derivative.

40. The method of claim 33, wherein the control is a normal cell.

41. The method of claim 33, wherein the control is a set of data from normal cells.

42. The method of claim 33, wherein the control is a physical genome map.
The method of claim 33, wherein the disorder is cancer.

43. The method of claim 33, wherein the difference in the methylation pattern is an increase in a total level of methylation.

44. The method of claim 33, wherein the difference in the methylation pattern is a decrease in a total level of methylation.

45. The method of claim 33, wherein the difference in the methylation pattern is a difference is location of methylation or type of methylation.

46. A method for assessing the efficacy of a therapeutic treatment, comprising:
determining a methylation pattern of a nucleic acid molecule in a biological sample from a subject prior to and after the therapeutic treatment, and
comparing the methylation pattern prior to the therapeutic treatment with the methylation pattern after the therapeutic treatment, wherein a difference of the methylation pattern of the nucleic acid molecule as a result of the therapeutic treatment is an indicator of the efficacy of the therapeutic treatment.

47. The method of claim 46, wherein the difference in the methylation pattern of the nucleic acid molecule is a decrease in a total level of methylation.

48. The method of claim 46, wherein the difference in the methylation pattern of the nucleic acid molecule is an increase in a total level of methylation.

49. The method of claim 46, wherein the difference in the methylation pattern of the nucleic acid molecule is a difference in location or type of methylation.

50. The method of claim 46, wherein the therapeutic treatment is an anti-cancer agent.

51. The method of claim 46, wherein the therapeutic treatment includes administration of an inhibitor of methyltransferase.

52. The method of claim 51, wherein the inhibitor of methyltransferase is selected from the group consisting of 5-azacytidine, 5-aza-2′deoxycytidine, 5,6-dihydro-5-azacytidine, 5-fluorocytidine and 5-fluoro-2′deoxycytidine.

53. A system for optically analyzing a nucleic acid molecule comprising:
an optical source for emitting optical radiation of a known wavelength;
an interaction station for receiving the optical radiation in an optical path and for receiving the nucleic acid molecule that is exposed to the optical radiation to produce detectable signals;
dichroic reflectors in the optical path for creating at least two separate wavelength bands of the detectable signals;
optical detectors constructed to detect radiation including the signals resulting from interaction of the nucleic acid molecule with the optical radiation; and
a processor constructed and arranged to analyze the nucleic acid molecule based on the detected radiation including the signals,
wherein the nucleic acid molecule is labeled according to its methylation status.

54. The method of claim 53, wherein the nucleic acid molecule is labeled by exposing it to a methylase and an S-adenosyl methionine derivative.

55. The method of claim 54, wherein the S-adenosyl methionine derivative is an aziridine derivative.

56. The system of claim 54, wherein the S-adenosyl methionine derivative comprises a label selected from the group consisting of a fluorescent molecule, a chemiluminescent molecule, a radioisotope, an enzyme substrate, a biotin molecule, an avidin molecule, an electrically charged transducing molecule, a nuclear magnetic resonance molecule, a semiconductor nanocrystal, an electromagnetic molecule, an electrically conducting particle, a ligand, a microbead, a magnetic bead, a Qdot, a chromogenic substrate, an affinity molecule, a protein, a peptide, a nucleic acid, a carbohydrate, an antigen, a hapten, an antibody, an antibody fragment, and a lipid.

57. The method of claim 53, wherein the nucleic acid molecule is labeled by exposing it an methylation-specific antibody or antibody fragment.

58. The method of claim 57, wherein the antibody or antibody fragment is conjugated to a label selected from the group consisting of a fluorescent molecule, a chemiluminescent molecule, a radioisotope, an enzyme substrate, a biotin molecule, an avidin molecule, an electrically charged transducing molecule, a nuclear magnetic resonance molecule, a semiconductor nanocrystal, an electromagnetic molecule, an electrically conducting particle, a ligand, a microbead, a magnetic bead, a Qdot, a chromogenic substrate, an affinity molecule, a protein, a peptide, a nucleic acid, a carbohydrate, an antigen, a hapten, an antibody, an antibody fragment, and a lipid.

59. The method of claim 53, wherein the nucleic acid molecule is labeled by exposing it to a methylated nucleic acid binding protein.

60. The method of claim 59, wherein the methylated nucleic acid binding protein is conjugated to a label selected from the group consisting of a fluorescent molecule, a chemiluminescent molecule, a radioisotope, an enzyme substrate, a biotin molecule, an avidin molecule, an electrically charged transducing molecule, a nuclear magnetic resonance molecule, a semiconductor nanocrystal, an electromagnetic molecule, an electrically conducting particle, a ligand, a microbead, a magnetic bead, a Qdot, a chromogenic substrate, an affinity molecule, a protein, a peptide, a nucleic acid, a carbohydrate, an antigen, a hapten, an antibody, an antibody fragment, and a lipid.

61. The method of claim 53, wherein the nucleic acid molecule is a non in vitro amplified nucleic acid molecule.

62. The system of claim 53, wherein the interaction station includes a slit having a slit width in the range of 1 nm to 500 nm and producing a localized radiation spot.

63. The system of claim 62, wherein the slit width is in the range of 10 nm to 100 nm.

64. The system of claim 62, wherein further comprising a microchannel arranged with the slit to produce the localized radiation spot, the microchannel being constructed to receive and advance the polymer units through the localized radiation spot.

65. The system of claim 64, further comprising a polarizer, wherein the optical source includes a laser constructed to emit a beam of radiation and the polarizer is arranged to polarize the beam prior to reaching the slit.

66. The system of claim 65, wherein the polarizer is arranged to polarize the beam in parallel to the width of the slit.

67. A method for analyzing a nucleic acid molecule comprising:
generating optical radiation of a known wavelength to produce a localized radiation spot;
passing a labeled nucleic acid molecule through a microchannel;
irradiating the labeled nucleic acid molecule at the localized radiation spot;
sequentially detecting radiation resulting from interaction of the labeled nucleic acid with the optical radiation at the localized radiation spot; and
analyzing the labeled nucleic acid molecule based on the detected radiation, wherein the nucleic acid molecule is labeled according to its methylation status.

68. The method of claim 67, further comprising employing an electric field to pass the nucleic acid molecule through the microchannel.

69. The method of claim 67, wherein the detecting includes collecting the signals over time while the nucleic acid molecule is passing through the microchannel.

70. The method of claim 67, wherein the nucleic acid molecule is labeled by exposing it to a methylase and an S-adenosyl methionine derivative.

71. The method of claim 67, wherein the S-adenosyl methionine derivative is an aziridine derivative.

72. The method of claim 70, wherein the S-adenosyl methionine derivative is conjugated to a label selected from the group consisting of a fluorescent molecule, a chemiluminescent molecule, a radioisotope, an enzyme substrate, a biotin molecule, an avidin molecule, an electrically charged transducing molecule, a nuclear magnetic resonance molecule, a semiconductor nanocrystal, an electromagnetic molecule, an electrically conducting particle, a ligand, a microbead, a magnetic bead, a Qdot, a chromogenic substrate, an affinity molecule, a protein, a peptide, a nucleic acid, a carbohydrate, an antigen, a hapten, an antibody, an antibody fragment, and a lipid.

73. The method of claim 67, wherein the nucleic acid molecule is labeled by exposing it to a methylation specific antibody or antibody fragment.

74. The method of claim 73, wherein the methylation-specific antibody or antibody fragment is conjugated to a label selected from the group consisting of a fluorescent molecule, a chemiluminescent molecule, a radioisotope, an enzyme substrate, a biotin molecule, an avidin molecule, an electrically charged transducing molecule, a nuclear magnetic resonance molecule, a semiconductor nanocrystal, an electromagnetic molecule, an electrically conducting particle, a ligand, a microbead, a magnetic bead, a Qdot, a chromogenic substrate, an affinity molecule, a protein, a peptide, a nucleic acid, a carbohydrate, an antigen, a hapten, an antibody, an antibody fragment, and a lipid.

75. The method of claim 67, wherein the nucleic acid molecule is labeled by exposing it to a methylated nucleic acid binding protein.

76. The method of claim 75, wherein the methylated nucleic acid binding protein is conjugated to a label selected from the group consisting of a fluorescent molecule, a chemiluminescent molecule, a radioisotope, an enzyme substrate, a biotin molecule, an avidin molecule, an electrically charged transducing molecule, a nuclear magnetic resonance molecule, a semiconductor nanocrystal, an electromagnetic molecule, an electrically conducting particle, a ligand, a microbead, a magnetic bead, a Qdot, a chromogenic substrate, an affinity molecule, a protein, a peptide, a nucleic acid, a carbohydrate, an antigen, a hapten, an antibody, an antibody fragment, and a lipid.

77. The method of claim 67, wherein the nucleic acid molecule is a non in vitro amplified nucleic acid molecule.

78. A method for analyzing a single nucleic acid molecule, comprising:
exposing a nucleic acid molecule to a labeled sequence-specific methylase and an S-adenosyl methionine derivative,
allowing the labeled sequence-specific methylase to bind to the nucleic acid molecule with the S-adenosyl methionine labeled derivative and label the nucleic acid molecule, and
determining a labeling pattern in the nucleic acid molecule using a linear polymer analysis system,
wherein the labeling pattern is indicative of a methylation pattern of the nucleic acid molecule.

79. The method of claim 78, wherein the nucleic acid molecule is a non in vitro amplified nucleic acid molecule.

80. The method of claim 78, wherein the S-adenosyl methionine derivative is labeled with a detectable label.

81. The method of claim 78, wherein the nucleic acid molecule is genomic DNA.

82. The method of claim 78, wherein the S-adenosyl methionine labeled derivative is an aziridine derivative.

83. The method of claim 78, wherein the labeling pattern in the nucleic acid molecule is determined using a method selected from the group consisting of Gene Engine™, optical mapping, and DNA combing.

84. The method of claim 78, wherein the nucleic acid molecule is exposed to a demethylating enzyme in an amount effective to demethylate the nucleic acid molecule, prior to exposure to the labeled sequence-specific methylase and the S-adenosyl methionine derivative.

85. The method of claim 78, wherein the nucleic acid molecule is exposed to a station to produce a signal arising from the nucleotide modification, and detecting the signal using a detection system.

86. The method of claim 78, wherein the labeled sequence specific methylase comprises a label selected from the group consisting of a fluorescent molecule, a chemiluminescent molecule, a radioisotope, an enzyme substrate, a biotin molecule, an avidin molecule, an electrically charged transducing molecule, a nuclear magnetic resonance molecule, a semiconductor nanocrystal, an electromagnetic molecule, an electrically conducting particle, a ligand, a microbead, a magnetic bead, a Qdot, a chromogenic substrate, an affinity molecule, a protein, a peptide, a nucleic acid, a carbohydrate, an antigen, a hapten, an antibody, an antibody fragment, and a lipid.

87. The method of claim 85, wherein the detection system is selected from the group consisting of a fluorescent detection system, an electrical detection system, a photographic film detection system, a chemiluminescent detection system, an enzyme detection system, an atom force microscopy (AFM) detection system, a scanning tunneling microscopy (STM) detection system, an optical detection system, a nuclear magnetic resonance (NMR) detection system, a near field detection system, a total internal reflection (TIR) system and a electromagnetic detection system.

88. The method of claim 78, further comprising labeling the nucleic acid molecule with a backbone label.

89. The method of claim 78, further comprising comparing the methylation pattern with a normal methylation pattern.

90. The method of claim 89, wherein the normal methylation pattern is determined from a normal subject.

91. The method of claim 89, wherein the normal methylation pattern is determined from a physical genome map.

92. A method for analyzing a single nucleic acid molecule, comprising:
exposing a nucleic acid molecule to a sequence-specific methylase and a labeled S-adenosyl methionine,
allowing the sequence-specific methylase to label the nucleic acid molecule with the labeled S-adenosyl methionine, and
determining a labeling pattern in the nucleic acid molecule using a linear polymer analysis system,
wherein the labeling pattern is indicative of a methylation pattern of the nucleic acid molecule.

93. The method of claim 92, wherein the nucleic acid molecule is a non in vitro amplified nucleic acid molecule.

94. The method of claim 92, wherein the nucleic acid molecule is DNA or RNA.

95. The method of claim 94, wherein the DNA is genomic DNA.

96. The method of claim 92, wherein the labeling pattern in the nucleic acid molecule is determined using a method selected from the group consisting of Gene Engine™, optical mapping, and DNA combing.

97. The method of claim 92, wherein the nucleic acid molecule is exposed to a demethylating enzyme in an amount effective to demethylate the nucleic acid molecule, prior to exposure to the sequence-specific methylase and the labeled S-adenosyl methionine.

98. The method of claim 1, further comprising, after determining the labeling pattern in the nucleic acid molecule,
exposing the nucleic acid molecule to a demethylating enzyme in an amount effective to demethylate the nucleic acid molecule,
re-exposing the nucleic acid molecule to a sequence-specific methylase and a labeled S-adenosyl methionine,
allowing the sequence-specific methylase to re-label target nucleotides in the nucleic acid molecule with the labeled S-adenosyl methionine, and
determining a labeling pattern in the nucleic acid molecule using a linear polymer analysis system.

99. The method of claim 98, wherein the labeling patterns prior to exposure to the demethylating enzyme and following the exposure to the demethylating enzyme are compared.

100. The method of claim 92, wherein the nucleic acid molecule is exposed to a station to produce a signal arising from the nucleotide modification, and detecting the signal using a detection system.

101. The method of claim 92, wherein the labeled S-adenosyl methionine comprises a label selected from the group consisting of a fluorescent molecule, a chemiluminescent molecule, a radioisotope, an enzyme substrate, a biotin molecule, an avidin molecule, an electrically charged transducing molecule, a nuclear magnetic resonance molecule, a semiconductor nanocrystal, an electromagnetic molecule, an electrically conducting particle, a ligand, a microbead, a magnetic bead, a Qdot, a chromogenic substrate, an affinity molecule, a protein, a peptide, a nucleic acid, a carbohydrate, an antigen, a hapten, an antibody, an antibody fragment, and a lipid.

102. The method of claim 100, wherein the detection system is selected from the group consisting of a fluorescent detection system, an electrical detection system, a photographic film detection system, a chemiluminescent detection system, an enzyme detection system, an atom force microscopy (AFM) detection system, a scanning tunneling microscopy (STM) detection system, an optical detection system, a nuclear magnetic resonance (NMR) detection system, a near field detection system, a total internal reflection (TIR) system and a electromagnetic detection system.

103. The method of claim 92, further comprising labeling the nucleic acid molecule with a backbone label.

104. The method of claim 92, further comprising comparing the methylation pattern with a normal methylation pattern.

105. The method of claim 104, wherein the normal methylation pattern is determined from a normal subject.

106. The method of claim 104, wherein the normal methylation pattern is determined from a physical genome map.
